(19) United States
(12) Patent Application Publication
(10) Pub. No.: US 2023/0149336 A1
(43) Pub. Date: May 18, 2023

(54) PREPARATION FOR USE AS ANTIOXIDANT

(71) Applicant: Evonik Operations GmbH, Essen (DE)

(72) Inventors: Rosario LIZIO, Muenster (DE); Michael SCHWARM, Alzenau (DE); Albino CARRIZZO, Castello del Matese (IT); Carmine VECCHIONE, Villapiana Scalo (IT)

(73) Assignee: Evonik Operations GmbH, Essen (DE)

(21) Appl. No.: 17/907,645

(22) PCT Filed: Mar. 24, 2021
(86) PCT No.: PCT/EP2021/057618

§ 371 Date: Sep. 28, 2022

(30) Foreign Application Priority Data
Apr. 1, 2020 (WO) 20167501.4

(51) Int. Cl. A61K 31/202; A23L 33/00; A23L 33/12; A23L 33/105; A61K 31/198; A61P 9/08; A61P 39/06; A61K 31/7048; A61K 36/185; A61K 36/45

(52) U.S. Cl. CPC A61K 31/202; A23L 33/40; A23L 33/12; A23L 33/105; A61K 31/198; A61P 9/08; A61P 39/06; A61K 31/7048; A61K 36/185; A61K 36/45

(57) ABSTRACT

A composition for use as antioxidant, where the composition has at least one polyunsaturated fatty acid component and at least one anthocyanin component. The polyunsaturated fatty acid component is an amino acid salt of the omega-3 fatty acids eicosapentaenoic acid (EPA) or docosahexaenoic acid (DHA). The anthocyanin component is cyanidin-3-glucoside, delphinidin-3-glucoside, malvidin-3-galactoside, peonidin-3-galactoside, or malvidin-3-glucoside. A method for treating a disease with the composition. A method of providing an antioxidant to a subject with the composition.

The current invention is related to a composition for use as antioxidant, wherein the composition comprises at least one polyunsaturated fatty acid component selected from an amino acid salt of the omega-3 fatty acids eicosapentaenoic acid (EPA) or docosahexaenoic acid (DHA) and one or more of the following anthocyanins: cyanidin-3-glucoside, delphinidin-3-glucoside, malvidin-3-galactoside, peonidin-3-galactoside, malvidin-3-glucoside.

Cardiovascular diseases including myocardial infarction (Ml), coronary artery diseases (CAD) and stroke remain the leading cause of death worldwide both in developed and developing countries. The development of major cardiovascular diseases is associated early in the process with the induction of an endothelial dysfunction characterized by a reduced formation of vasoprotective factors including nitric oxide (NO), increasing pro-oxidant factors and often also by the development of endothelium-dependent contracting responses.

Dietary intake of omega-3 fatty acids, namely alpha-linoleic acid (ALA), EPA and DHA, is beneficial for human health, in particular with respect to e.g. the amelioration of rheumatoid arthritis and reduction of cardiovascular disease risk factors (Balk EM, Lichtenstein A H, Nutrients 2017, 9(8); Calder P C, Biochim Biophys Acta 2015, 1851(4):469-484). Various seafood products are a source of dietary EPA/DHA, but their consumption is often not sufficient to meet the recommended dietary allowance (typically 500 mg EPA and DHA per day) (Papanikolaou Y et. al. 3^{rd}, NutrJ 2014, 13:31). This gap is closed by the widespread use of dietary supplements or fortified foods containing omega-3 fatty acids (Clarke TC et al. Natl Health Stat Report 2015(79):1-16). Dietary supplements are concentrated sources of nutrients or other substances with a nutritional or physiological effect, whose purpose is to supplement the normal diet (www.efsa.europa.eu/en/topics/topic/food-supplements). For example, omega-3 fatty acid supplements often contain either triglycerides or omega-3 ethyl esters of EPA/DHA from fish oil, krill oil, or algae.

Omega-3 fatty acids in general have anti-inflammatory, cardio- and neuroprotective effects (Schunck W H et al. Pharmacol Ther 2018, 183:177-204). Their modes of action involve e.g. direct scavenging of reactive oxygen species, alteration of cell membrane fluidity, which subsequently affects cellular signaling events, modulation of the activity of transcription factors such as PPARγ and NFkappaB that orchestrate the biosynthesis of pro- and anti-inflammatory cytokines, and competitive exclusion of substrates that are converted to proinflammatory mediators by cyclooxygenases and lipoxygenases. Although their potential beneficial effects include reduction of susceptibility to ventricular arrhythmia, antithrombogenic and antioxidant effect, retardation of the atherosclerotic plaque growth, anti-inflammatory effect, and mild hypotensive effect, the mechanisms by which they exert their cardiovascular protection have not been clarified.

Since daily consumption of these omega-3 sources with food or nutritional supplements is limited, it's important to assure maximum bioavailability of these fatty acids. Bioavailability of hydrophobic nutrients in the digestive system is often low and represents a challenge especially for supplements, because they are frequently consumed independently from a meal in the form of capsules or pills. Secretion of digestive fluids (bile acids, phospholipids, lipases) is hardly or not at all induced in the fasted state, which results in incomplete enzymatic hydrolysis of fats and oils, low solubilization and bioavailability.

Additional bioavailability challenges arise, when advanced formulation technologies are used to skip parts of the digestive systems in order to release omega-3 fatty acids in the lower part of the digestive system, e.g. in the small or large intestine. Capsules or tablets coated with respective release polymers can be used for this purpose. In these systems, the above mentioned, natural solubilization mechanisms are less effective, which reduces bioavailability and has to be compensated by appropriate measures.

In the context of the present invention the term PUFA is used interchangeably with the term polyunsaturated fatty acid and defined as follows: Fatty acids are classified based on the length and saturation characteristics of the carbon chain. Short chain fatty acids have 2 to about 6 carbons and are typically saturated. Medium chain fatty acids have from about 6 to about 14 carbons and are also typically saturated. Long chain fatty acids have from 16 to 24 or more carbons and may be saturated or unsaturated. In longer chain fatty acids there may be one or more points of unsaturation, giving rise to the terms “monounsaturated” and “polyunsaturated,” respectively. In the context of the present invention long chain polyunsaturated fatty acids having 20 or more carbon atoms are designated as polyunsaturated fatty acids or PUFAs.

PUFAs are categorized according to the number and position of double bonds in the fatty acids according to well established nomenclature. There are two main series or families of LC-PUFAs, depending on the position of the double bond closest to the methyl end of the fatty acid: The omega-3 series contains a double bond at the third carbon, while the omega-6 series has no double bond until the sixth carbon. Thus, docosahexaenoic acid (DHA) has a chain length of 22 carbons with 6 double bonds beginning with the third carbon from the methyl end and is designated “22:6 n-3” (all-cis-4,7,10,13,16,19-docosahexaenoic acid). Another important omega-3 PUFA is eicosapentaenoic acid (EPA) which is designated “20:5 n-3” (all-cis-5,8,11,14,17-eicosapentaenoic acid). An important omega-6 PUFA is arachidonic acid (ARA) which is designated “20:4 n-6” (all-cis-5,8,11,14-eicosatetraenoic acid).

Other omega-3 PUFAs include: Eicosatrienoic acid (ETE) 20:3 (n-3) (all-cis-11,14,17-eicosatrienoic acid), Eicosatetraenoic acid (ETA) 20:4 (n-3) (all-cis-8,11,14,17-eicosatetraenoic acid), Heneicosapentaenoic acid (HPA) 21:5 (n-3) (all-cis-6,9,12,15,18-heneicosapentaenoic acid), Docosapentaenoic acid (Clupanodonic acid) (DPA) 22:5 (n-3) (all-cis-7,10,13,16,19-docosapentaenoic acid), Tetracosapentaenoic acid 24:5 (n-3) (all-cis-9,12,15,18,21-tetracosapentaenoic acid), Tetracosahexaenoic acid (Nisinic acid) 24:6 (n-3) (all-cis-6,9,12,15,18,21-tetracosahexaenoic acid).

Other omega-6 PUFAs include: Eicosadienoic acid 20:2 (n-6) (all-cis-11,14-eicosadienoic acid), Dihomo-gamma-linolenic acid (DGLA) 20:3 (n-6) (all-cis-8,11,14-eicosatrienoic acid), Docosadienoic acid 22:2 (n-6) (all-cis-13,16-docosadienoic acid), Adrenic acid 22:4 (n-6) (all-cis-7,10,13,16-docosatetraenoic acid), Docosapentaenoic acid (Osbond acid) 22:5 (n-6) (all-cis-4,7,10,13,16-docosapentaenoic acid), Tetracosatetraenoic acid 24:4 (n-6) (all-cis-9,12,15,18-tetracosatetraenoic acid), Tetracosapentaenoic acid 24:5 (n-6) (all-cis-6,9,12,15,18-tetracosapentaenoic acid).

Preferred omega-3 PUFAs used in the embodiments of the present invention are docosahexaenoic acid (DHA) and eicosapentaenoic acid (EPA).

Various approaches have been developed to solve the bioavailability problem, either by formulation, chemical modification of omega-3 fatty acids or both. One promising approach is the hydrolysis and subsequent saponification of omega-3 fatty acid esters, which mimics part of the natural digestive process and thereby increases solubility. WO2016102323A1 describes compositions comprising polyunsaturated omega-3 fatty acid salts that can be stabilized against oxidation.

During the last years, epidemiological studies have identified a relationship between diet and CVD, there is still considerable scientific uncertainty about the relationship between specific dietary components and cardiovascular risk (Schmitt and Ferro 2013 British Journal of Clinical Pharmacology, 75: 585-87; Carrizzo et al. 2019 Hypertension, 73: 449-57). A promising dietary group for cardiovascular protection are polyphenols, especially flavonoids, as they are inversely associated with blood pressure and lower risk of hypertension (Godos, et al., 2019 Food & Nutrition Research, 61: 1-21). On this regard, anthocyanins, natural pigments belonging to the flavonoid family are widely distributed in the human diet such as beans, fruits, vegetables, and red wine (Khoo et al. 2017 Food & Nutrition Research, 61:1-21). Actually, it is well-accepted that these natural products present in fruits and plant-derived-foods are relevant because of their potential health-promoting effects, as suggested by the available experimental and epidemiological evidence (Wallace 2011a). For this reason, interest in the biochemistry and biological effects of anthocyanin compounds has increased substantially during the last decade. It has been reported that anthocyanins exert positive effects on human health reducing inflammatory processes and counteracting oxidative stress (de Pascual-Teresa, Moreno and Garcia-Viguera 2010 Int J Mol Sci, 11: 1679-703), improving the blood lipid profile, inhibiting the growth of cancerous cells (Hou 2003 Curr Mol Med, 3: 149-59.) and to owning anti-obesity effects (Tsuda et al. 2003 J Nutr, 133: 2125-30). With regard to CVD, anthocyanins from blueberries or red wine showed an improvement in flow mediated dilation (FMD), and augmentation index in human, as well as NO-dependent vessel relaxation in mice (Andriambeloson, et al., 1998; Curtis, et al., 2019 J Nutr, 139: 2266-71; Rodriguez-Mateos, et al., 2019). Although all its beneficial properties, the possible direct action of anthocyanins on the vasculature, both at functional and molecular levels, remains completely unknown.

Anthocyanins are water-soluble vacuolar pigments that may appear red, purple or blue, depending on the surrounding pH-value. Anthocyanins belong to the class of flavonoids, which are synthesized via the phenylpropanoid pathway. They occur in all tissues of higher plants, mostly in flowers and fruits and are derived from anthocyanidins by addition of sugars. Anthocyanins are glycosides of flavylium salts. Each anthocyanin thus comprises three component parts: the hydroxylated core (the aglycone); the saccharide unit; and the counterion. Anthocyanins are naturally occurring pigments present in many flowers and fruit and individual anthocyanins are available commercially as the chloride salts, e.g. from Polyphenols Laboratories AS, Sandnes, Norway. The most frequently occurring anthocyanins in nature are the glycosides of cyanidin, delphinidin, malvidin, pelargonidin, peonidin and petunidin.

It is known that anthocyanins, especially resulting from fruit intake, have a wide range of biological activities, including antioxidant, anti-inflammatory, antimicrobial and anti-carcinogenic activities, improvement of vision, induction of apoptosis, and neuroprotective effects. Particularly suitable fruit sources for the anthocyanins are cherries, bilberries, blueberries, black currants, red currants, grapes, cranberries, strawberries, cowberries, elderberries, saskatoon berries and apples and vegetables such as red cabbage, black scented rice (especially the varieties Chakhao Poireiton and Chakhao Amubi), blue maize, winter barley, etc. (Benvenuti et al, Journal of Food Science, Vol, 69, Nr, 3, 2004; Escalante-Aburto et al., Journal of Chemistry, Volume 2016 and Diczhazi et al, Cereal Chemistry (2014), 91(2), 195-200). Bilberries, in particular Vaccinium myrtillus, and black currants, in particular Ribes nigrum, are especially suitable.

Although their beneficial action, it has been reported that anthocyanins frequently interact with other phytochemicals, exhibiting synergistic biological effects making contributions from individual components difficult to decipher. In fact, the majority of intervention studies investigating anthocyanins have used foods containing several types of polyphenols. Only few studies have been performed using compounds (i.e. Medox®) containing purified anthocyanins isolated from bilberries. On this regard, it has been demonstrated that anthocyanin supplementation for 3-weeks reduces several NF-kB-regulated pro-inflammatory chemokines and immunoregulatory cytokines (Karlsen, A. et al. 2007. ‘Anthocyanins inhibit nuclear factor-kappaB activation in monocytes and reduce plasma concentrations of pro-inflammatory mediators in healthy adults’, J Nutr, 137: 1951-4). Other studies showed an effect on HDL-C upregulation and LDL-C downregulation after 12-weeks of consumption (Qin, Y. et al. 2009. ‘Anthocyanin supplementation improves serum LDL- and HDL-cholesterol concentrations associated with the inhibition of cholesteryl ester transfer protein in dyslipidemic subjects’, American Journal of Clinical Nutrition, 90: 485-92). However, an interesting study did not find similar effects on blood lipids after 500 mg of anthocyanins (cyanidin 3-glucoside) for 12 weeks (Curtis, P. J. et al. 2009. ‘Cardiovascular disease risk biomarkers and liver and kidney function are not altered in postmenopausal women after ingesting an elderberry extract rich in anthocyanins for 12 weeks’, J Nutr, 139: 2266-71), and hypothesized that different anthocyanins may possess different bioactivities.

Bilberries contain diverse anthocyanins, including delphinidin and cyanidin glycosides and include several closely related species of the genus Vaccinium, including Vaccinium myrtillus (bilberry), Vaccinium uliginosum (bog bilberry, bog blueberry, bog whortleberry, bog huckleberry, northern bilberry, ground hurts), Vaccinium caespitosum (dwarf bilberry), Vaccinium deliciosum (Cascade bilberry), Vaccinium membranaceum (mountain bilberry, black mountain huckleberry, black huckleberry, twin-leaved huckleberry), Vaccinium ovalifolium (oval-leafed blueberry, oval-leaved bilberry, mountain blueberry, high-bush blueberry).

Dry bilberry fruits of V. myrtillus contain up to 10% of catechin-type tannins, proanthocyanidins, and anthocyanins. The anthocyanins are mainly glucosides, galactosides, or arabinosides of delphinidin, cyanidin, and—to a lesser extent—malvidin, peonidin, and petunidin (cyanidin-3 glucoside (C3G), delphinidin-3-O-glucoside (D3G), malvidin-3-O-glucoside (M3G), peonidin-3 glucoside and petunidin-3-O-glucoside). Flavonols include quercetin- and kaempferol-glucosides. The fruits also contain other phenolic compounds (e.g., chlorogenic acid, caffeic acid, o-, m-, and p-coumaric acids, and ferulic acid), citric and malic acids, and volatile compounds.

Black currant fruits (R. nigrum) contain high levels of polyphenols, especially anthocyanins, phenolic acid derivatives (both hydroxybenzoic and hydroxycinnamic acids), flavonols (glycosides of myricetin, quercetin, kaempferol, and isorhamnetin), and proanthocyanidins (between 120 and 166 mg/100 g fresh berries). The main anthocyanins are delphinidin-3-O-rutinoside (D3R) and cyanidin-3-O-rutinoside (C3R), but D3G and C3G are also found (Gafner, Bilberry-Laboratory Guidance Document 2015, Botanical Adulterants Program).

EP 1443948 A1 relates to a process for preparing a nutritional supplement (nutraceutical) comprising a mixture of anthocyanins from an extract of black currants and bilberries. Anthocyanins were extracted from cakes of fruit skin produced as the waste product in fruit juice pressing from V. myrtillus and R. nigrum. It could be shown that the beneficial effects of individual anthocyanins are enhanced if instead of an individual anthocyanin, a combination of different anthocyanins is administered orally, in particular a combination comprising both mono and disaccharide anthocyanins. It is thought that the synergistic effect arises at least in part from the different solubilities and different uptake profiles of the different anthocyanins.

In the context it was surprisingly found that polyunsaturated fatty acid components selected from ethyl esters of the omega-3 fatty acids eicosapentaenoic acid (EPA) or docosahexaenoic acid (DHA) or amino acid salts of EPA or DHA exert an important vasorelaxant effect of mice resistance arteries. It could be demonstrated for the first time that an omega-3 lysine complex (AvailOm®) is able to evoke a direct endothelial vasorelaxation through the activation of nitric oxide dependent mechanism. In addition, it is able to significantly improve the endothelial impairment and the oxidative stress evoked by oxidized LDL. Though a vascular reactivity study and molecular analysis, it could be shown that AvailOm® exerts a direct vascular action inducing a dose-dependent vasorelaxation, which is dependent to AMPK/eNOS axis. Moreover, the combination of AvailOm®, using a ratio 1:1, with most potent anthocyanins involved in the modulation of vascular tone, Cyanidin-3-O-galactoside (C3-gal) or C3-gal plus Delphinidin-3-o-arabinoside (DP3-ara) in combination, significantly improve dose-dependent vasorelaxation and nitric oxide production.

Moreover, it was surprisingly found that the combination of AvailOm® with an anthocyanin mixture, which contains C3-glu+DP3-glu+MaI3-glu+MaI3-gal+PEO3-gal, maintaining a 1:6 ratio, was able to significantly improve endothelial dependent vasorelaxation and reduce the oxidative stress after ox-LDL treatment. It is important to emphasize that these are the first studies that investigate the possible vascular effect of the combination of omega-3 fatty acids and anthocyanins.

Therefore, the invention is related to a composition for use as antioxidant, wherein the composition comprises at least one polyunsaturated fatty acid component selected from an amino acid salt of the omega-3 fatty acids eicosapentaenoic acid (EPA) or docosahexaenoic acid (DHA) and one or more of the following anthocyanins: cyanidin-3-glucoside, delphinidin-3-glucoside, malvidin galactoside, peonidin-3-galactoside, malvidin-3-glucoside.

The omega-3 forms that are commonly used in food fortification or nutritional supplements are krill oil, fish oil, or ethyl esters derived from the former. Recently, a technology has been described to stabilize EPA/DHA free fatty acids with amino acids resulting in solid and somewhat inert salts of EPA/DHA that can be introduced into e.g. food or supplement preparations. WO2016102323A1 describes compositions comprising polyunsaturated omega-3 fatty acid salts that can be stabilized against oxidation. WO2017202935A1 discloses a method for preparing a composition comprising omega-3 fatty acid salts and amines wherein a paste comprising one or more omega-3 fatty acid(s), one or more basic amine(s) and 20% by weight or less water, based on the total weight of the paste, is kneaded until a homogenous paste is obtained.

Compositions comprising polyunsaturated fatty acids may be obtained from any suitable source material which, additionally, may have been processed by any suitable method of processing such source material. Typical source materials include any part of fish carcass, vegetables and other plants as well as material derived from microbial and/or algal fermentation. Typically, such material further contains substantial amounts of other naturally occurring fatty acids. Typical methods of processing such source materials may include steps for obtaining crude oils such as extraction and separation of the source material, as well as steps for refining crude oils such as settling and degumming, de-acidification, bleaching, and deodorization, and further steps for producing PUFA-concentrates from refined oils such as de-acidification, trans-esterification, concentration, and deodorization (cf. e.g. EFSA Scientific Opinion on Fish oil for Human Consumption). Any processing of source materials may further include steps for at least partially transforming PUFA-esters into the corresponding free PUFAs or inorganic salts thereof.

Salts of lysine with polyunsaturated fatty acids per se are known in the art (cf. EP 0734373 B1), and were described as “very thick transparent oils, which transform into solids of waxy appearance and consistency at low temperatures” (cf. EP 0734373 B1, page 1, lines 47 to 48). However, salts of PUFAs can be obtained via spray drying conditions as described in WO2016102323A1 and WO2016102316A1.

In a preferred embodiment of the present invention, the amount of polyunsaturated fatty acid is 65 weight % or less, preferably 60 weight % or less, more preferably between 40 and 55 weight-% with respect to the total weight of polyunsaturated fatty acid salt.

It is preferred, when the omega-3 fatty acid salts have an organic counter ion selected from lysine, arginine, ornithine, choline and mixtures of the same.

It is particularly preferred to use fatty acid salts comprising EPA and DHA and having an organic counter ion selected from lysine, arginine and ornithine. The lysine salt of EPA and DHA are even more preferred.

In a preferred embodiment, the composition comprises at least two of the following anthocyanins: cyanidin-3-glucoside, delphinidin-3-glucoside, malvidin-3-galactoside, peonidin-3-galactoside, malvidin-3-glucoside.

It is further preferred, if the composition comprises the following anthocyanins: cyanidin glucoside, delphinidin-3-glucoside, malvidin-3-galactoside, peonidin-3-galactoside and malvidin-3-glucoside.

Information on anthocyanin content on different fruits can be found in the literature, such as for black currant red currant, black chokeberry, bilberry, cowberry, elderberry, (Benvenuti, S. et al. (2006). ‘Polyphenols, Anthocyanins, Ascorbic Acid, and Radical Scavenging Activity of Rubus, Ribes, and Aronia’, Journal of Food Science, Vol, 69, Nr, 3, 2004; Kăhkŏnen, M. P. et al. 2003. ‘Berry anthocyanins: isolation, identification and antioxidant activities’, J Sci Food Agric 83:1403-1411; Wu X et al. 2004 ‘Characterization of anthocyanins and proanthocyanidins in some cultivars of Ribes, Aronia, and Sambucus and their antioxidant capacity’, J, Agric, Food Chem, 52, 7846-7856), strawberry, sweet cherry and sour cherry (Jakobek L. et al. 2007. ‘Flavonols, Phenolic Acids and Antioxidant Activity of Some Red Fruits’, Deutsche Lebensmittel-Rundschau, 103, Jahrgang, Heft 2, 2007), wild blueberries and Saskatoon berries (Hosseinian FS et al. 2007 ‘ Saskatoon and wild blueberries have higher anthocyanin contents than other Manitoba berries’, Journal of Agricultural and Food Chemistry, 55(26), 10832-10838), rhubarb petioles (Takeoka, G. R. et al. 2013.‘Antioxidant activity, phenolic and anthocyanin contents of various rhubarb (Rheum spp.) varieties’, International Journal of Food Science and Technology, 48(1), 172-178), black scented rice Chakhao Poireton, Chakhao Amubi (Asem, I. D. et al. 2015. ‘Anthocyanin content in the black scented rice (Chakhao): its impact on human health and plant defense’, Symbiosis (2015), 66(1), 47-54).

High amounts of cyanidin-3-glucoside are especially present in the following fruits: blackberries, elderberries, sweet cherry, blue maize, Korean colored rice (Heuginju), Saskatoon berries.

High amounts of delphinidin-3-glucoside are present in black currant, wild blueberries, Saskatoon berries.

High amounts of malvidin-3-galactoside are present in bilberries, Saskatoon berries, wild blueberries.

High amounts of peonidin-3-galactoside are present in wild blueberries, Saskatoon berries.

High amounts of malvidin-3-glucoside are present in bilberries, wild blueberries, Saskatoon berries.

Preferred mixture comprises fruits or fruit extracts selected from black currants, bilberries, blackberries, elderberries, sweet cherry, Saskatoon berries, wild blueberries. Such fruit mixtures cover a mixture of the relevant anthocyanins cyanidin-3-glucoside, delphinidin-3-glucoside, malvidin-3-galactoside, peonidin-3-galactoside, malvidin-3-glucoside.

It is particularly preferred to provide mixtures with similar amounts of the beneficial anthocyanins, to ensure maximum antioxidant capacity. Therefore, in an advantageous configuration of the present invention, the mixture comprises fruits or fruit extracts from bilberries and wild blueberries.

In an alternative configuration, the mixture comprises fruit extracts from black currants and bilberries.

In a preferred embodiment, the mixture comprises the specific fruits in defined ratios (in weight-%):bilberries:wild blueberries in ratios between 10:1 and 1:1, preferably between 8:1 and 2:1, more preferably of 4:1.

In a preferred embodiment, the composition is for preventing or treating a disease or disorder selected from cardiovascular diseases, preferably atherosclerosis, hypertension, stroke, diabetes-related cardiovascular disfunctions, ischemia/reperfusion injury, hypercholesterolemia, coronary artery disease, chronic obstructive pulmonary disease (COPD).

In another preferred embodiment, the composition is for preventing or treating a disease or disorder in connection with stress and low mental performance, preferably Burnout, low cognitive performance, bad sleep quality, and stress situations in general.

The invention also relates to a composition comprising at least one omega-3 fatty acid amino acid salt, comprising eicosapentaenoic acid (EPA) and docosahexaenoic acid (DHA) and one or more of the following anthocyanins: cyanidin-3-glucoside, delphinidin-3-glucoside, malvidin-3-galactoside, peonidin-3-galactoside, malvidin-3-glucoside.

It is preferred, when the omega-3 fatty acid amino acid salt has an organic counter ion selected from lysine, arginine, ornithine and mixtures of the same, preferably lysine.

In a preferred embodiment, the composition comprises fruits or extracts selected from the following: bilberries, cranberries, cowberries, lingonberries, red, yellow and green apple, aronia, black chokeberry, black scented rice (Chakhao Poireton, Chakhao Amubi) and winter barley, preferably black chokeberry, bilberries and cowberries. It is preferred when the composition comprises fruits or fruit extracts selected from the following: bilberries and wild blueberries.

In a preferred embodiment, the composition comprises the omega-3 fatty acid amino acid salt and fruits or fruit extracts of bilberries and wild blueberries in a ratio (weight-%) between 10:1 and 1:1, preferably between 8:1 and 2:1, more preferably of 4:1.

### WORKING EXAMPLES

Materials:

The omega-3 lysine salt (AvailOm®) was obtained from Evonik Nutrition & Care GmbH, Darmstadt (Germany) and contains around 32 weight-% of L-lysine and around 65 weight-% of polyunsaturated fatty acids. The major polyunsaturated fatty acids in the composition are the omega-3 fatty acids Eicosapentaenoic acid (C20:5w3c) (EPA) and Docosahexaenoic acid (C22:6w3c) (DHA), summing up to around 58 weight-% of the composition. The composition also contains minor amounts of Docosaenoic acid isomer (incl. erucic acid) (C22:1), Docosapentaenoic acid (C22:5w3c) and of the omega-6 fatty acids Arachidonic acid (C20:4w6) and Docosatetraenoic acid (C22:4w6c). The single ω-3 Fatty Acids (ω-3 FA) and L-Lysin were obtained from Evonik Nutrition & Care GmbH, Darmstadt (Germany), the ω-3 Ethyl Ester (ω-3 EE) were obtained from Solutex GC S. L., Madrid (Spain). oxLDL has been acquired from Thermo Fisher. All the inhibitors, powders and solvents necessary for the preparation of the buffers were purchased by Sigma-Aldrich.

Healthberry 865® (HB) is a dietary supplement consisting of 17 purified anthocyanins (all glycosides of cyanidin, peonidin, delphinidin, petunidin, and malvidin) isolated from black currant (Ribes nigrum) and bilberries (Vaccinium myrtillus) and was obtained from Evonik Nutrition & Care GmbH, Darmstadt (Germany). The major anthocyanins contained in the berry extract used are cyanidin-3-glucoside, cyanidin-3-rutinoside, delphinidin-3-glucoside, delphinidin-3-rutinoside, cyanidin-3-galactoside and delphinidin-3-galactoside. The amount of anthocyanin citrate is at least 25 weight-% of the composition. The composition is prepared from black currants and bilberries by a process comprising the steps of alcoholic extraction of black currants and bilberries, purification via chromatography, mixing of the extracts with maltodextrin citrate and water and spray-drying of the mixture. The product composition contains extracts of black currants and bilberries mixed in a weight ratio of around 1:1.

The single anthocyanins, Delphinidin-3-rutinoside (D3-rut), Cyanidin-3-rutinoside (C3-rut), Delphinidin-3-glucoside (DP3-glu), Cyanidin-3-glucoside (C3-glu), Petunidin-3-glucoside (PT3-glu), Delphinidin-3-galactoside (DP3-gal), Peonidin-3-galactoside (PEO3-gal), Delphinidin-3-arabinoside (DP3-ara), Malvidin-3-galactoside (MAL3-gal), Malvidin-3-glucoside (MAL3-glu), Cyanidin-3-galactoside (C3-gal), Cyanidin-3-arabinopyranoside (C3-arapy) were obtained from Polyphenols AS, Sandnes (Norway).

Experimental Animals

All experiments involving animals were conformed to the Guide for the Care and Use of Laboratory Animals published by the US National Institutes of Health (NIH Publication No. 85-23, revised 2011) and were approved by review board. Wild-type C57BL/6 mice (weighing— 25 g) (Jackson Laboratories, Bar Harbor, Me., USA) have been used to perform vascular reactivity and molecular studies.

Vascular Reactivity Studies

Second-order branches of the mesenteric arterial tree were removed from mice to perform vascular studies. Vessels were placed in a wire or pressure myograph system filled with Krebs solution maintained at pH 7.4 at 37° C. in oxygenated (95% O₂/5% CO₂). First, an analysis of vascular reactivity curves was performed. In particular, vasoconstriction was assessed with 80 mmol/L of KCl or with increasing doses of phenylephrine (from 10⁻⁹ M to 10⁻⁶ M) in control conditions. Endothelium-dependent and-independent relaxations were assessed by measuring the dilatory responses of mesenteric arteries to cumulative concentrations of acetylcholine (from 10-9 M to 10-6 M) or nitroglycerine (from 10-9 M to 10-6 M) respectively, in vessels precontracted with phenylephrine at the dose necessary to obtain a similar level of precontraction in each ring (80% of initial KCl-evoked contraction). Caution was taken to avoid endothelial damage; functional integrity was reflected by the response to acetylcholine (from 10⁻⁹ M to 10⁻⁶ M).

Vascular responses were then tested administering increasing doses of Healthberry 865®-865 or single anthocyanins. Some experiments were performed in presence of selective inhibitors, such as phosphatidylinositol-4,5-bisphosphate 3-kinase inhibitor (LY274002, 10 μM, 1 h), Akt inhibitor (Akt inh, 1 μM, 1 h) or the NOS inhibitor N-w-nitro-1-arginine methyl ester (L-NAME, 300 μM, 30 min) before data for dose-response curves were obtained.

Evaluation of NO Production by DAF

Production of NO was assessed as previously described (Carrizzo et al. 2016). AvailOm® (100 μg/mL) or acetylcholine (10-6 M) was administered to the mesenteric artery in the last 30 min of 4-amino-5-methylamino-2,7,-difluorofluorescein diacetate (DAF-FM) incubation, alone and after 20 min exposure to L-NAME (300 umol/L, 30 min). Mesenteric segments were cut in 5-μm thick sections, observed under a fluorescence microscope, subsequently counterstained with haematoxylin and eosin and observed under a light microscope.

Analysis of Total ROS Production

Dihydroethidium (DHE, Life Technologies) was used to evaluate production of reactive oxygen species (ROS) in mouse mesenteric arteries, as previously described. Briefly, vessels were incubated with 5 μM of DHE for 20 min and subsequently observed under a fluorescence microscope (Zeiss). Images were acquired by a digital camera system (Olympus Soft Imaging Solutions). A second, estimation of total ROS production in mouse vessels was performed with the membrane-permeable fluorescent probe an analog of 2,7-Dichlorodihydrofluorescein (DCDHF), Dihydrorhodamine 123 (DHR123) (Invitrogen). After treatment, vessels were incubated with Krebs solution containing 5 μM DHR123 for 30 min at 37° C., and then washed two times with PBS prior to fluorescence measurement using a fluorescence microplate reader (TECAN infinite 200 Pro).

Statistical Analysis

Data are presented as mean±SEM. Statistical analysis was performed by 2-way ANOVA followed by Bonferroni post hoc test. Repeated measurements were analysed by One-way ANOVA followed Bonferroni post-hoc test. Differences were considered to be statistically significant at p<0.05.

### Example 1: AvailOm® Evokes a Direct Vasorelaxant Action on Mice Mesenteric Arteries

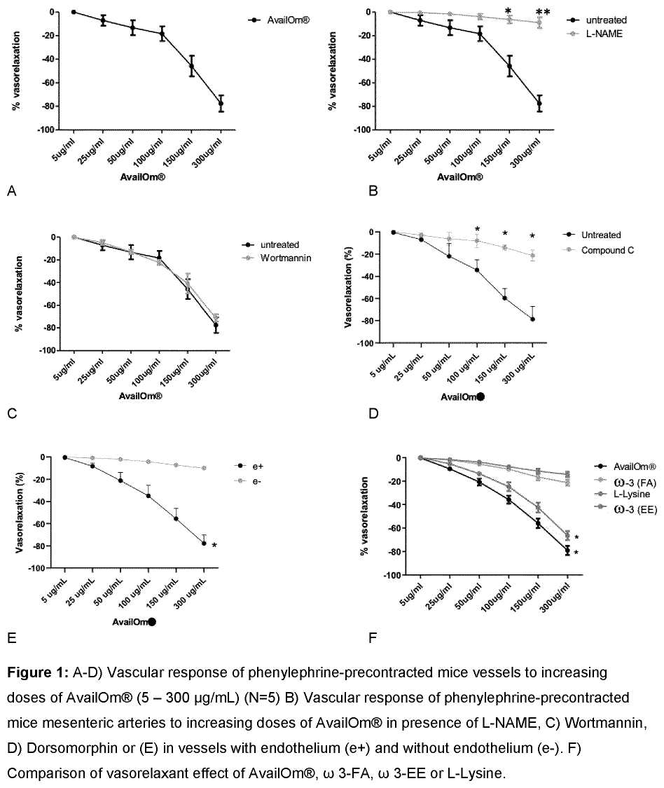

To assess the possible direct vascular action of AvailOm®, vascular reactivity studies on mice vessels were performed, administering increasing doses of AvailOm® (5-300 ug/mL) on pre-constricted mice mesenteric arteries, considering the concept that alteration of vascular response of resistance arteries reflects in an important contribution to the development of cardiovascular complications. The data demonstrate that AvailOm® exerts a direct dose-response vasorelaxant action (FIG. 1A). This effect is due to the stimulation of nitric oxide production, since the inhibition of eNOS enzyme, by L-NAME, completely abolishes this effect (FIG. 1B). Considering one of the major enzymes involved in eNOS activation, its vascular action in presence of phosphoinositide 3-kinase (PI3K) inhibitor was assessed, demonstrating that this mechanism is not involved in its direct vascular action (FIG. 1C). Interestingly, in presence of selective AMPK inhibitor, dorsomorphin, AvailOm® completely loses its capability to evoke endothelial-dependent vasorelaxation (FIG. 1D). Study performed in absence of endothelial layer demonstrate that endothelium represents the main target of the compound (FIG. 1E). Assessment of vascular response to L-Lysine did not evoke any vasorelaxant effect (FIG. 1F). This result was similarly to that observed with ω-3-FA (FIG. 1F). In contrast, assessment of vasorelaxant properties of ω-3-EE was able to induces a dose-dependent vasorelaxation, however, the effect shown for AvailOm® was tendentially stronger (FIG. 1F).

FIG. 1 shows in A-D) vascular response of phenylephrine-precontracted mice vessels to increasing doses of AvailOm® (5-300 μg/mL) (N=5) B) Vascular response of phenylephrine-precontracted mice mesenteric arteries to increasing doses of AvailOm® in presence of L-NAME, C) Wortmannin, D) Dorsomorphin or (E) in vessels with endothelium (e+) and without endothelium (e−). F) Comparison of vasorelaxant effect of AvailOm®, ω3-FA, ω3-EE or L-Lysine. Statistical analyses were performed using two-way ANOVA followed Bonferroni post-hoc test. *p<0.05;**p<0.01, ***p<0.001.

### Example 2: AvailOm® Prevents Vascular Oxidative Stress Damage Induced by ox-LDL

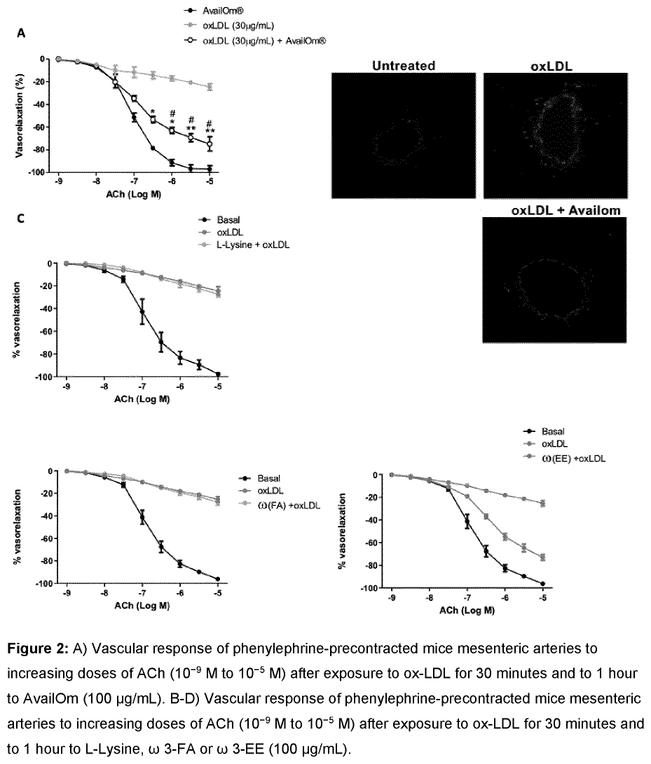

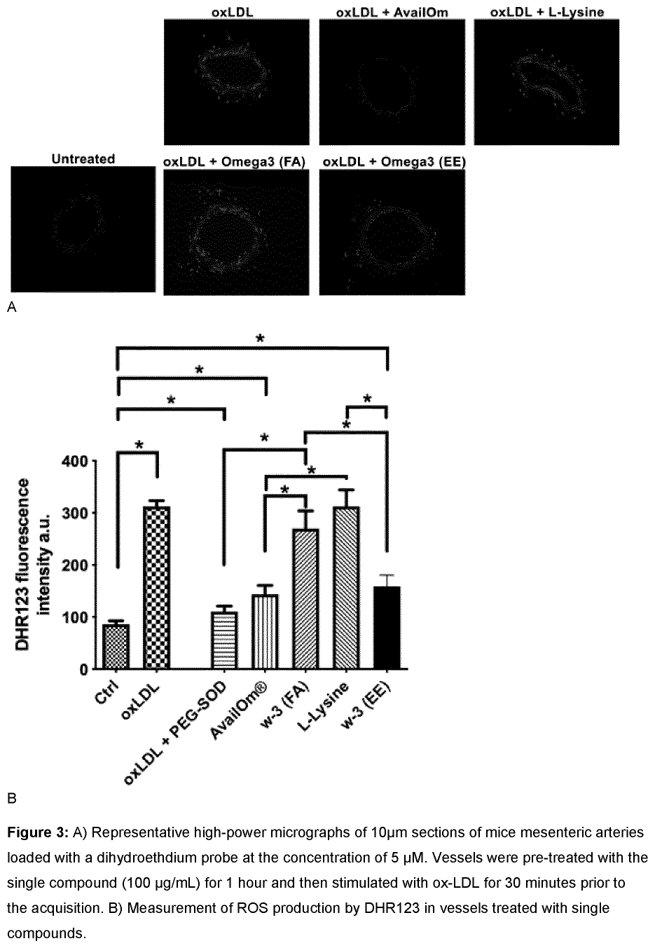

Subsequently the possible effect of AvailOm® on oxidative stress induced by oxLDL was assessed. As reported in FIG. 2, a pre-treatment with AvailOm® (100 μg/mL) of vessels exposed to ox-LDL leads to a significant protection from oxidative stress as showed by endothelial response to ACh (FIG. 2A). Of note, the evaluation of oxidative stress by dihydroethidium, demonstrates a complete protection from oxLDL-evoked oxidative stress (FIG. 2B). Interestingly, the assessment of the protection from oxLDL-evoked vascular oxidative stress, revealed that EE form of ω-3 is the major component that owns the cardiovascular beneficial properties (FIG. 2C-D-E). The qualitative and quantitative assessment of oxidative stress by dihydroethidium and DHR123, respectively, demonstrate that ω-3-EE reproduce a similar effect of AvailOm® alone, however, AvailOm® having a slightly stronger effect to protect from vascular oxidative stress in vitro (FIG. 3A-B).

FIG. 2 shows in A) vascular response of phenylephrine-precontracted mice mesenteric arteries to increasing doses of ACh (10-9 M to 10-5 M) after exposure to ox-LDL for 30 minutes and to 1 hour to AvailOm (100 μg/mL). B-D) Vascular response of phenylephrine-precontracted mice mesenteric arteries to increasing doses of ACh (10-9 M to 10-5 M) after exposure to ox-LDL for 30 minutes and to 1 hour to L-Lysine, ω3-FA or ω3-EE (100 μg/mL). Statistical analyses were performed using two-way ANOVA followed Bonferroni post-hoc test. *p<0.05; **p<0.01, ***p<0.001.

FIG. 3 shows in A) representative high-power micrographs of 10 μm sections of mice mesenteric arteries loaded with a dihydroethdium probe at the concentration of 5 μM. Vessels were pre-treated with the single compound (100 μg/mL) for 1 hour and then stimulated with ox-LDL for 30 minutes prior to the acquisition. B) Measurement of ROS production by DHR123 in vessels treated with single compounds. Statistical analyses were performed using one-way ANOVA followed Bonferroni post-hoc test. *p<0.05; **p<0.01, ***p<0.001.

### Example 3: AvailOm® in Combination with Most Powerful Anthocyanins Exerts Most Potent Vasorelaxant Effect

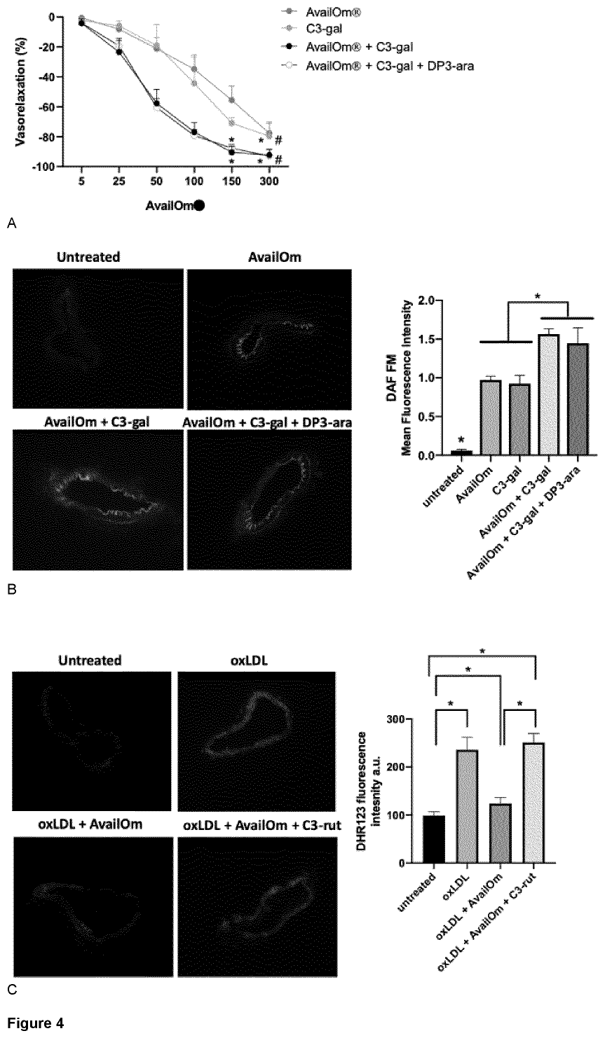

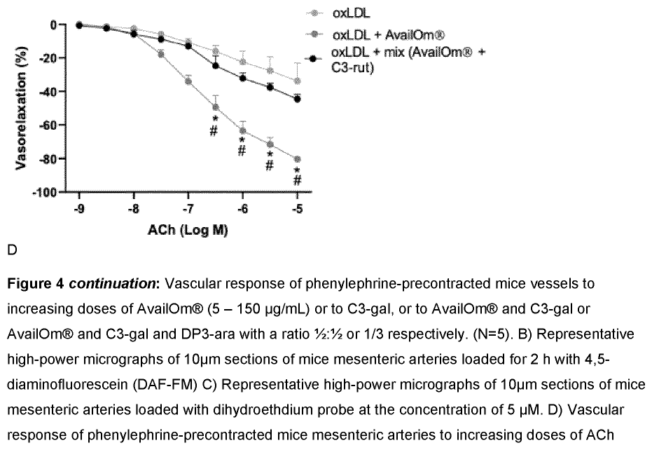

In a next step, the possible vascular action of AvailOm® in combination with different anthocyanins Cyanidin-3-O-galactoside (C3-gal) or C3-gal plus Delphinidin-3-o-arabinoside (DP3-ara) was assessed, maintaining a ratio ½:½, maintaining the same overall amount of the substance to be tested. The data demonstrate that in presence of both C3-gal and C3-gal with DP3-ara, AvailOm® is able to exert a most powerful vasorelaxant effect, which can be seen in a significant improvement of endothelial dependent vasorelaxation at 50, 100 and 150 μg/mL in comparison to AvailOm® alone (FIG. 4A). This shows that the effect is not just additive, but has a clear synergy between the omega-3 fatty acid salt and the anthocyanins used.

The assessment of nitric oxide production by DAF-FM revealed both in presence of C3-gal and C3-gal with DP3-ara a significant improvement of NO production in comparison to AvailOm® or C3-gal alone (100 μg/mL) (FIG. 4B). Measurement of antioxidative action of AvailOm® with C3-rut, the most powerful antioxidant anthocyanin revealed that the protective action of AvailOm® from ox-LDL evoked oxidative stress was significantly reduced in comparison to AvailOm® alone.

FIG. 4 shows vascular response of phenylephrine-precontracted mice vessels to increasing doses of AvailOm® (5-150 μg/mL) or to C3-gal, or to AvailOm® and C3-gal or AvailOm® and C3-gal and DP3-ara with a ratio ½:½ or ⅓ respectively. (N=5). B) Representative high-power micrographs of 10 μm sections of mice mesenteric arteries loaded for 2 h with 4,5-diaminofluorescein (DAF-FM) reveal nitric oxide production after treatment with AvailOm® or single combination. Bar graph shows the mean fluorescence intensity of N=4 section for each compound. C) Representative high-power micrographs of 10 μm sections of mice mesenteric arteries loaded with dihydroethdium probe at the concentration of 5 μM. Vessels were pre-treated with ox-LDL, oxLDL plus AvailOm® or with oxLDL plus Availom® mixed with C3-rut (½:½) and D) measurement of ROS production by DHR123. D) Vascular response of phenylephrine-precontracted mice mesenteric arteries to increasing doses of ACh (10-9 to M 10-5 M) after exposure to ox-LDL for 30 minutes and exposed to AvailOm® or AvailOm® mixed with C3-rut.

### Example 4: AvailOm® in Combination with Anthocyanin Mix Exerts a Potent Vasorelaxant Effect

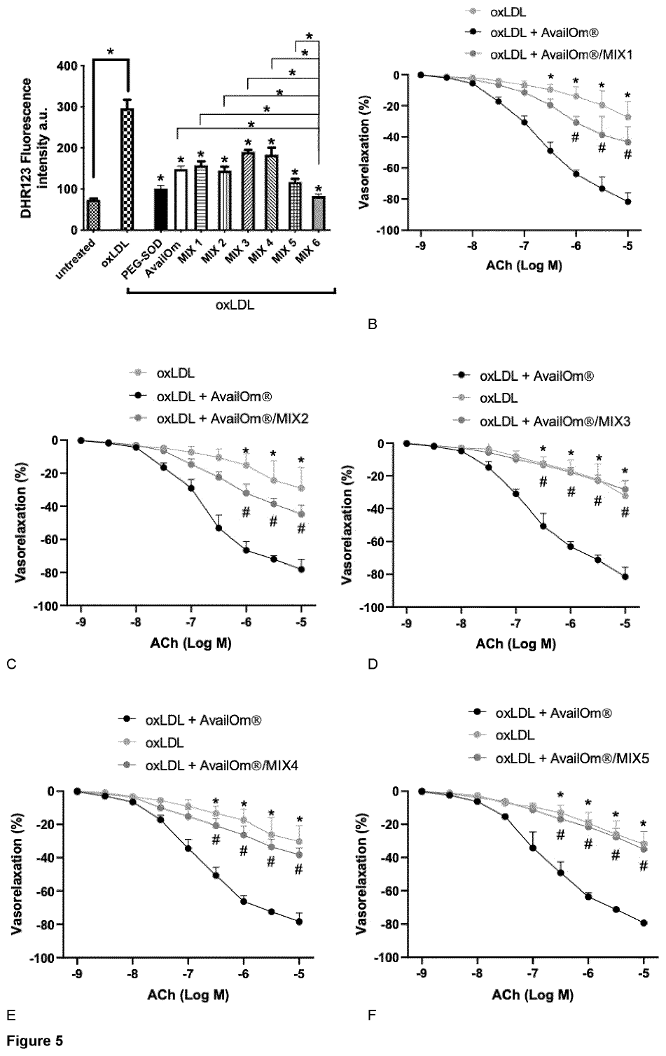

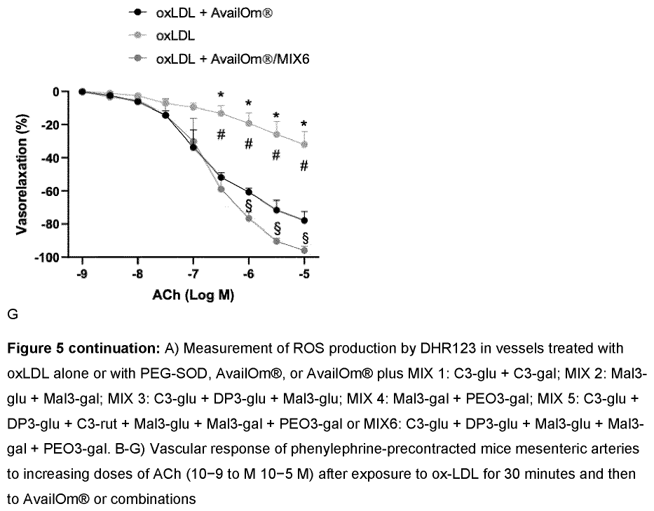

The possible action of AvailOm® in combination with different anthocyanins' mixtures on ROS production was analyzed. First of all, the measurement of oxLDL evoked ROS production showed that AvailOm® plus MIX6 (C3-glu+DP3-glu+MaI3-glu+MaI3-gal+PEO3-gal), respecting a ratio of 1:6 of each product, was able to significantly reduce the oxidative stress with a major degree respecting to AvailOm® alone or AvailOm® in combination with MIX 1 (C3-glu+C3-gal), MIX 2 (MaI3-glu+MaI3-gal), MIX 3 (C3-glu+DP3-glu+MaI3-glu), MIX 4 (MaI3-gal+PEO3-gal) or MIX 5:C3-glu+DP3-glu+C3-rut+MaI3-glu+MaI3-gal+PEO3-gal (FIG. 5A). Interestingly, the evaluation of endothelial vasorelaxation under oxLDL-evoked oxidative stress revealed that AvailOm in combination with MIX6 exert the major protection from ROS evoked endothelial dysfunction (FIG. 5B-G) demonstrating an unexpected synergistic effect with AvailOm®.

FIG. 5 shows in A) measurement of ROS production by DHR123 in vessels treated with ox-LDL alone or with PEG-SOD, AvailOm®, or AvailOm® plus MIX 1: C3-glu+C3-gal; MIX 2: MaI3-glu+MaI3-gal; MIX 3: C3-glu+DP3-glu+MaI3-glu; MIX 4: MaI3-gal+PEO3-gal; MIX 5: C3-glu+DP3-glu+C3-rut+MaI3-glu+MaI3-gal+PEO3-gal or MIX6: C3-glu+DP3-glu+MaI3-glu+MaI3-gal+PEO3-gal. Statistical analyses were performed using one-way ANOVA followed Bonferroni post-hoc test. *p<0.05. B-G) Vascular response of phenylephrine-precontracted mice mesenteric arteries to increasing doses of ACh (10-9 to M 10-5 M) after exposure to ox-LDL for 30 minutes and then to AvailOm® for 1 hour alone or AvailOm® in combination with MIX1, MIX2, MIX3, MIX4, MIX5 or MIX6.*P<0.05 vs oxLDL+AvailOm®. #p<0.05 oxLDL+AvailOm®; § p<0.05 vs ox-LDL+AvailOm®.

### Example 5: The Antioxidant Vascular Action of Healthberry 865® is Due to the Combination of the Anthocyanins Contained

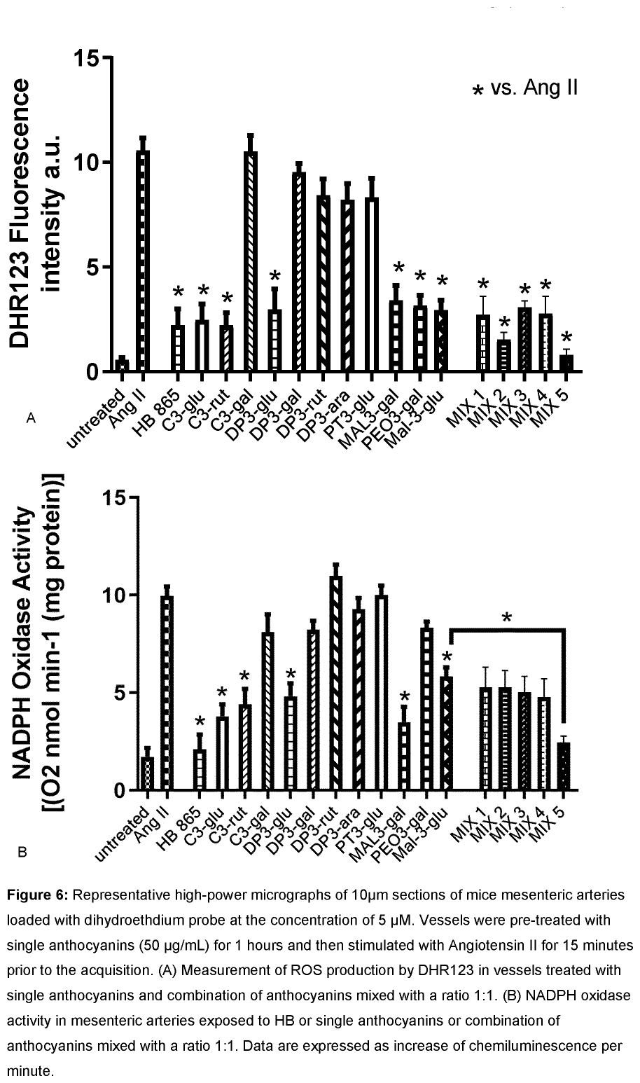

Previously few studies have reported an antioxidant activity of Healthberry 865® in human subjects (Karlsen et al. 2007). To investigate the capability of Healthberry 865® and the single anthocyanins contained on the modulation of oxidative stress, several methodological approaches were performed measuring both, total anti reactive oxygen species (ROS) capacity and their specific action on the modulation of the main machinery of ROS production, the activity of NADPH oxidase enzyme. The studies performed on mice mesenteric arteries revealed that Healthberry 865® owns an important anti-oxidative action, as shown by the significant reduction of Angiotensin II-induced ROS formation (FIG. 6). The antioxidant vascular actions of single anthocyanins contained in Healthberry 865®: Delphinidin-3-rutinoside (D3-rut), Cyanidin-3-rutinoside (C3-rut), Delphinidin glucoside (DP3-glu), Cyanidin-3-glucoside (C3-glu), Petunidin-3-glucoside (PT3-glu), Delphinidin galactoside (DP3-gal), Peonidin-3-galactoside (PEO3-gal), Delphinidin-3-arabinoside (DP3-ara), Malvidin-3-galactoside (MAL3-gal), Malvidin-3-glucoside (MAL3-glu), Cyanidin-3-galactoside (C3-gal) and Cyanidin-3-arabinopyranoside (C3-arapy) were analyzed. A deeper analysis, using single anthocyanins revealed that C3-glu, C3-rut, DP3-glu, MAL3-gal, PEO3-gal, MaI-3-glu are able to reproduce the antioxidant action of Healthberry 865®. Accordingly, the biochemical measurement of ROS generation by DHR1,2,3 probe confirm the results obtained with DHE (FIG. 6B).

Moreover, the analysis of NADPH oxidase (NOX) activity after stimulation with Angiotensin II, a gold-standard inducer of NOX activation was performed. The results showed that C3-glu, C3-rut, DP3-glu, MAL3-gal, PEO3-gal, MAL3-glu are able to reduce NOX activity. However, these single anthocyanins resulted in a smaller reduction than evoked by Healthberry 865® (FIG. 6C). In fact, C3-glu and MAL3-glu resulted to be the most powerful anthocyanins closer to the potent effect of Healthberry 865®.

To evaluate the role on oxidative stress, the action of further mixtures was analyzed: MIX 1: C3-glu+C3-gal; MIX 2: MaI3-glu+MaI3-gal; MIX 3: C3-glu+DP3-glu+MaI3-glu; MIX 4: MaI3-gal+PEO3-gal; MIX 5: C3-glu+DP3-glu+C3-rut+MaI3-glu+MaI3-gal+PEO3-gal. Interestingly, the measurement of both total ROS production and that of NADPH oxidase activity revealed highest efficacy of MIX 5.

FIG. 6 shows representative high-power micrographs of 10 μm sections of mice mesenteric arteries loaded with dihydroethdium probe at the concentration of 5 μM. Vessels were pre-treated with single anthocyanins (50 μg/mL) for 1 hours and then stimulated with Angiotensin II for 15 minutes prior to the acquisition. (A) Measurement of ROS production by DHR123 in vessels treated with single anthocyanins and combination of anthocyanins mixed with a ratio 1:1. (B) NADPH oxidase activity in mesenteric arteries exposed to HB or single anthocyanins or combination of anthocyanins mixed with a ratio 1:1. Data are expressed as increase of chemiluminescence per minute.

### Example 6: Mixture of Different Fruits for an Optimized Ratio of Anthocyanins with Antioxidant Activities in Combination with AvailOm®

In order to achieve an optimal ratio of all anthocyanins, which have a strong vasorelaxant effect, literature values for the content of the single anthocyanins in specific fruits were compared. Since it is postulated that the beneficial anthocyanins shall be present in a nearly equimolar ratio, the fruits with the highest amounts of the respective anthocyanins were combined in different ratios to achieve balanced ratios of the anthocyanins cyanidin-3-glucoside, delphinidin-3-glucoside, malvidin-3-galactoside, peonidin-3-galactoside, malvidin-3-glucoside.

The content of anthocyanins was analyzed in detail for black currant, red currant, black chokeberry bilberry, cowberry, elderberry (Benvenuti et al., 2004; Kăhkŏnen et al., 2003; Wu et al., 2004), strawberry, sweet cherry and sour cherry (Jakobek et al., 2007), wild blueberries and Saskatoon berries (Hosseinian et al., 2007).

By mixing fruits with high amounts of the desired anthocyanins, the following contents of the specific anthocyanins were achieved:

TABLE 1
mixture of bilberry and wild blueberry in the ratio of 1:1

| | Total amount | Total amount | |
|---|---|---|---|
| | in mixture | (weight-%/total | |
| Anthocyanin | (mg/100 g) | anthocyanin amount) | Ratio |
| cyanidin-3-glucoside | 1123 | 33 | 16 |
| delphinidin-3-glucoside | 431 | 12 | 6 |
| malvidin-3-galactoside | 127 | 4 | 2 |
| peonidin-3-galactoside | 65 | 2 | 1 |
| malvidin-3-glucoside | 222 | 6 | 3 |
| others | 1483 | 43 | 21 |
| Sum | 3451 | 100 | |

After mixing the desired berries in the ratio of 1:1, the specific anthocyanins are present in different amounts in the mixture, differing by a factor of up to 16.

By mixing fruits with high amounts of the desired anthocyanins in an optimized ratio, the following contents of the specific anthocyanins were achieved:

TABLE 2
mixture of bilberry and wild blueberry in the ratio of 4:1

| | Total amount | Total amount | |
|---|---|---|---|
| | in mixture | (weight-%/total | |
| Anthocyanin | (mg/100 g) | anthocyanin amount) | Ratio |
| cyanidin-3-glucoside | 223 | 9 | 2, 3 |
| delphinidin-3-glucoside | 308 | 13 | 3, 1 |
| malvidin-3-galactoside | 149 | 6 | 1, 5 |
| peonidin-3-galactoside | 152 | 6 | 1, 6 |
| malvidin-3-glucoside | 280 | 11 | 2, 9 |
| others | 1334 | 55 | 13, 6 |
| Sum | 2446 | 100 | |

After mixing the desired berries in the ratio (weight-%) of 4:1, the specific anthocyanins are present in similar amounts in the mixture, differing by a factor of less than 2. This corresponds to the mixing ratio of anthocyanins from the previous experiments.

### REFERENCES

Andriambeloson E., Kleschyov A. L., Muller B., Beretz A., Stoclet J. C., Andriantsitohaina R. Nitric oxide production and endothelium-dependent vasorelaxation induced by wine polyphenols in rat aorta. Br. J. Pharmacol. 1997; 120:1053-1058.
Asem, I. D., Imotomba, R. K., Mazumder, P. B., Laishram, J. M., 2015. ‘Anthocyanin content in the black scented rice (Chakhao):its impact on human health and plant defense’, Symbiosis (2015), 66(1), 47-54.
Balk E M, Lichtenstein A H, ‘Omega-3 fatty acids and cardiovascular disease’, Nutrients 2017, 9(8);
Benvenuti, S., Pellati, F., Melegari, M., Bertelli, D. (2006). ‘Polyphenols, Anthocyanins, Ascorbic Acid, and Radical Scavenging Activity of Rubus, Ribes, and Aronia’, Journal of Food Science, Vol, 69, Nr, 3, 2004
Calder P C, ‘Marine omega-3 fatty acids and inflammatory processes: Effects, mechanisms and clinical relevance.’ Biochim Biophys Acta 2015, 1851(4):469-484.
Carrizzo, A., M. Ambrosio, A. Damato, M. Madonna, M. Storto, L. Capocci, P. Campiglia, E. Sommella, V. Trimarco, F. Rozza, R. Izzo, A. A. Puca, and C. Vecchione. 2016. ‘Morus alba extract modulates blood pressure homeostasis through eNOS signaling’, Mol Nutr Food Res, 60: 2304-11.
Clarke T. C., Black L. I., Stussman B. J., Barnes P. M., Nahin R. L., Trends in the use of complementary health approaches among adults: United States, 2002-2012., Natl Health Stat Report 2015(79):1-16.
Curtis, P. J., P. A. Kroon, W. J. Hollands, R. Walls, G. Jenkins, C. D. Kay, and A. Cassidy. 2009. ‘Cardiovascular disease risk biomarkers and liver and kidney function are not altered in postmenopausal women after ingesting an elderberry extract rich in anthocyanins for 12 weeks’, J Nutr, 139: 2266-71.
de Pascual-Teresa, S., D. A. Moreno, and C. Garcia-Viguera. 2010. ‘Flavanols and anthocyanins in cardiovascular health: a review of current evidence’, Int J Mol Sci, 11: 1679-703.
Diczhäzi, I. and Kursinszki, L., 2014 ‘Anthocyanin Content and Composition in Winter Blue Barley Cultivars and Lines’, Cereal Chemistry, 91, 2, (195-200).
DiNicolantonio, J. J., A. K. Niazi, M. F. McCarty, J. H. O'Keefe, P. Meier, and C. J. Lavie. 2014. ‘Omega-3s and cardiovascular health’, Ochsner J, 14: 399-412.
Dyerberg, J., H. O. Bang, E. Stoffersen, S. Moncada, and J. R. Vane. 1978. ‘Eicosapentaenoic acid and prevention of thrombosis and atherosclerosis?’, Lancet, 2: 117-9.
Escalante-Aburto, A., Ponce-Garcïa, N., Ramirez-Wong, B., Torres-Chavez, P. I., de Dios Figueroa-Cärdenas, J., Gutiërrez-Dorado, R., 2016. ‘Specific Anthocyanin Contents of Whole Blue Maize Second-Generation Snacks: An Evaluation Using Response Surface Methodology and Lime Cooking Extrusion’ Journal of Chemistry, Volume 2016
Galle, J., J. Bengen, P. Schollmeyer, and C. Wanner. 1995. ‘Impairment of endothelium-dependent dilation in rabbit renal arteries by oxidized lipoprotein(a). Role of oxygen-derived radicals’, Circulation, 92: 1582-9.
Goode, G. K., S. Garcia, and A. M. Heagerty. 1997. ‘Dietary supplementation with marine fish oil improves in vitro small artery endothelial function in hypercholesterolemic patients: a double-blind placebo-controlled study’, Circulation, 96: 2802-7.
Hosseinian F S1, Beta T. 2007 ‘ Saskatoon and wild blueberries have higher anthocyanin contents than other Manitoba berries’, Journal of Agricultural and Food Chemistry, 55(26), 10832-10838.
Hou, D. X. 2003. ‘Potential mechanisms of cancer chemoprevention by anthocyanins’, Curr Mol Med, 3: 149-59.
Iwamatsu, K., S. Abe, H. Nishida, M. Kageyama, T. Nasuno, M. Sakuma, S. Toyoda, and T. Inoue. 2016. ‘Which has the stronger impact on coronary artery disease, eicosapentaenoic acid or docosahexaenoic acid?’, Hypertens Res, 39: 272-5.
Jakobek L., Seruga, M., Novak, I, Medividovic-Kasonavic, M. 2007. ‘Flavonols, Phenolic Acids and Antioxidant Activity of Some Red Fruits’, Deutsche Lebensmittel-Rundschau, 103, Jahrgang, Heft 2, 2007
Jensen, G. S., X. Wu, K. M. Patterson, J. Barnes, S. G. Carter, L. Scherwitz, R. Beaman, J. R. Endres, and A. G. Schauss. 2008. ‘In vitro and in vivo antioxidant and anti-inflammatory capacities of an antioxidant-rich fruit and berry juice blend. Results of a pilot and randomized, double-blinded, placebo-controlled, crossover study’, J Agric Food Chem, 56: 8326-33.
Jiang, S., T. Li, Z. Yang, W. Yi, S. Di, Y. Sun, D. Wang, and Y. Yang. 2017. ‘AMPK orchestrates an elaborate cascade protecting tissue from fibrosis and aging’, Ageing Res Rev, 38: 18-27.
Kăhkŏnen, M. P., Heinămăki, J., 011ilainen, V. and Heinonen, M 2003. ‘Berry anthocyanins: isolation, identification and antioxidant activities’, J Sci Food Agric 83:1403-1411
Karlsen, A., L. Retterstol, P. Laake, I. Paur, S. K. Bohn, L. Sandvik, and R. Blomhoff. 2007. ‘Anthocyanins inhibit nuclear factor-kappaB activation in monocytes and reduce plasma concentrations of pro-inflammatory mediators in healthy adults’, J Nutr, 137: 1951-4.
Khoo, H. E., A. Azlan, S. T. Tang, and S. M. Lim. 2017. ‘Anthocyanidins and anthocyanins: colored pigments as food, pharmaceutical ingredients, and the potential health benefits’, Food & Nutrition Research, 61: 1-21.
Li, H., and U. Forstermann. 2009. ‘Prevention of atherosclerosis by interference with the vascular nitric oxide system’, Curr Pharm Des, 15: 3133-45.
McVeigh, G. E., G. M. Brennan, G. D. Johnston, B. J. McDermott, L. T. McGrath, W. R. Henry, J. W. Andrews, and J. R. Hayes. 1993. ‘Dietary fish oil augments nitric oxide production or release in patients with type 2 (non-insulin-dependent) diabetes mellitus’, Diabetologia, 36: 33-8.
Papanikolaou Y et. al. U.S. adults are not meeting recommended levels for fish and omega-3 fatty acid intake: results of an analysis using observational data from NHANES 2003-2008., 3^{1d}, Nutr J 2014, 13:31.
Qin, Y., M. Xia, J. Ma, Y. Hao, J. Liu, H. Mou, L. Cao, and W. Ling. 2009. ‘Anthocyanin supplementation improves serum LDL- and HDL-cholesterol concentrations associated with the inhibition of cholesteryl ester transfer protein in dyslipidemic subjects’, American Journal of Clinical Nutrition, 90: 485-92.
Rodriguez-Mateos, A., Istas, G., Boschek, L., Feliciano, R. P., Mills, C. E., Boby, C., et al. (2019). Circulating anthocyanin metabolites mediate vascular benefits of blueberries: insights from randomized controlled trials, metabolomics, and nutrigenomics. J. Gerontol. A Biol. Sci. Med. Sci. [Epub ahead of print]
Schunck W. H., Konkel A., Fischer R., Weylandt K. H. Therapeutic potential of omega-3 fatty acid-derived epoxyeicosanoids in cardiovascular and inflammatory diseases., Pharmacol Ther 2018, 183:177-204.
Schmitt, J., and A. Ferro. 2013. ‘Nutraceuticals: is there good science behind the hype?’, British Journal of Clinical Pharmacology, 75: 585-87.
Shaughnessy, K. S., I. A. Boswall, A. P. Scanlan, K. T. Gottschall-Pass, and M. I. Sweeney. 2009. ‘Diets containing blueberry extract lower blood pressure in spontaneously hypertensive stroke-prone rats’, Nutr Res, 29: 130-8.
Takeoka, G. R., Dao, L., Harden, L., Pantoja, A., Kuhl, J. C. 2013.‘Antioxidant activity, phenolic and anthocyanin contents of various rhubarb (Rheum spp.) varieties’, International Journal of Food Science and Technology, 48(1), 172-178.
Takikawa, M., S. Inoue, F. Norio, and T. Tsuda. 2010. ‘Dietary anthocyanin-rich bilberry extract ameliorates hyperglycemia and insulin sensitivity via activation of AMP-activated protein kinase in diabetic mice’, J Nutr, 140: 527-33.
Tsuda, T., F. Norio, K. Uchida, H. Aoki, and T. Osawa. 2003. ‘Dietary cyanidin 3-O-beta-D-glucoside-rich purple corn color prevents obesity and ameliorates hyperglycemia in mice’, J Nutr, 133: 2125-30.
Wallace, T. C. 2011a. ‘Anthocyanins in Cardiovascular Disease’, Advances in Nutrition, 2: 1-7.2011b. ‘Anthocyanins in cardiovascular disease’, Advances in Nutrition, 2: 1-7.
Wang, X. M., H. Xiao, L. L. Liu, D. Cheng, X. J. Li, and L. Y. Si. 2016. ‘FGF21 represses cerebrovascular aging via improving mitochondrial biogenesis and inhibiting p53 signaling pathway in an AMPK-dependent manner’, Exp Cell Res, 346: 147-56.
Woodman, R. J., T. A. Mori, V. Burke, I. B. Puddey, A. Barden, G. F. Watts, and L. J. Beilin. 2003. ‘Effects of purified eicosapentaenoic acid and docosahexaenoic acid on platelet, fibrinolytic and vascular function in hypertensive type 2 diabetic patients’, Atherosclerosis, 166: 85-93.
Wu, X., J. Kang, C. Xie, R. Burris, M. E. Ferguson, T. M. Badger, and S. Nagarajan. 2010. ‘Dietary blueberries attenuate atherosclerosis in apolipoprotein E-deficient mice by upregulating antioxidant enzyme expression’, J Nutr, 140: 1628-32.
Zhang, Y., F. Lian, Y. Zhu, M. Xia, Q. Wang, W. Ling, and X. D. Wang. 2010. ‘Cyanidin-3-O-beta-glucoside inhibits LPS-induced expression of inflammatory mediators through decreasing IkappaBalpha phosphorylation in THP-1 cells’, Inflamm Res, 59: 723-30.

1. A composition for, wherein the composition comprises:
at least one polyunsaturated fatty acid component of an amino acid salt of eicosapentaenoic acid (EPA) and an amino acid salt of docosahexaenoic acid (DHA); and
at least one anthocyanin selected from the group consisting of cyanidin-3-glucoside, delphinidin-3-glucoside, malvidin-3-galactoside, peonidin-3-galactoside, and malvidin-3-glucoside.

2. The composition according to claim 1, wherein the amino acid salts have at least one organic counter ion selected from the group consisting of lysine, arginine, ornithine and mixtures thereof.

3. The composition according to claim 1, wherein the composition further comprises at least one fruit or fruit extract selected from the group consisting of black currants, bilberries, blackberries, elderberries, sweet cherry, Saskatoon berries, and wild blueberries.

4. The composition according to claim 3, wherein the composition further comprises at least one fruit or fruit extract selected from the group consisting of bilberries and wild blueberries.

5. The composition according to claim 1, wherein the composition further comprises an extract of black currants and bilberries.

6. (canceled)

7. A composition comprising:
an amino acid salt of, eicosapentaenoic acid (EPA) and an amino acid salt of docosahexaenoic acid (DHA); and
at least one anthocyanin selected from the group consisting of cyanidin-3-glucoside, delphinidin-3-glucoside, malvidin-3-galactoside, peonidin-3-galactoside, and malvidin-3-glucoside.

8. The composition of claim 7, wherein the amino acid salts have at least one organic counter ion selected from the group consisting of lysine, arginine, ornithine and mixtures thereof.

9. The composition according to claim 7, wherein the composition further comprises at least one fruit or fruit extract selected from the group consisting of bilberries and wild blueberries.

10. The composition according to claim 9, wherein the composition further comprises the fruits or fruit extracts of bilberries and wild blueberries in a ratio (weight-%) between 10:1 and 1:1.

11. A method of treating a disease, comprising:
administering the composition of claim 1 to a patient in need thereof, wherein the disease is cardiovascular disease, atherosclerosis, hypertension, stroke, diabetes-related cardiovascular disfunction, ischemia/reperfusion injury, hypercholesterolemia, coronary artery disease, or chronic obstructive pulmonary disease (COPD).

12. A method of providing an antioxidant to a subject, comprising:
administering the composition of claim 1 to a subject in need thereof.
